(19) United States
(12) Patent Application Publication
(10) Pub. No.: US 2005/0159897 A1
(43) Pub. Date: Jul. 21, 2005

(54) Postponing validation of speculative chromosomes

(76) Inventors: David M. Anderson, Fort Collins, CO (US); Tyson R. McGuffin, Fort Collins, CO (US); Thomas W. Chen, Fort Collins, CO (US)

Correspondence Address: HEWLETT PACKARD COMPANY, P O BOX 272400, 3404 E. HARMONY ROAD, INTELLECTUAL PROPERTY ADMINISTRATION, FORT COLLINS, CO, 80527-2400, US

(21) Appl. No.: 10/761,832

(22) Filed: Jan. 21, 2004

(51) Int. Cl. G06N 3/00; G06N 3/12; G06F 15/18; G06F 19/00; G01N 33/48; G01N 33/50

(52) U.S. Cl. 702/20; 706/13

(57) ABSTRACT

Systems and methods are disclosed for selecting a value set associated with a set of parameters. Real costs are generated for each of a plurality of first value sets represented as a plurality of real chromosomes. Speculative costs are generated for each of a plurality of second value sets represented as a plurality of speculative chromosomes that represent value set variations of the first value sets. At least one subsequent generation of speculative chromosomes is generated until a predetermined validation criteria has been satisfied. Upon achieving the predetermined validation criteria, real costs are generated for at least one speculative chromosome of the plurality of speculative chromosomes.

### CROSS REFERENCE TO RELATED APPLICATIONS

This application is related to the following commonly assigned co-pending patent applications entitled: “SPECULATION COUNT IN A GENETIC ALGORITHM,” Attorney Docket No. 200309413-1; “SPECULATIVE POOL,” Attorney Docket No. 200309414-1; “SYSTEMS AND METHODS FOR SELECTING A VALUE SET,” Attorney Docket No. 200309416-1, all of which are filed contemporaneously herewith and are incorporated herein by reference.

### BACKGROUND

Genetic algorithms are application technologies inspired by mechanisms of inheritance and evolution of living things. In the evolution of living things, genomic changes like crossovers of chromosomes and mutations of genes can occur when new individuals (children) are born from old individuals (parents). In a genetic algorithm, a candidate of a solution to an optimization problem is represented as a data structure, referred to as a chromosome. The data structure represents a plurality of variables or bits referred to as genes. Typically, a plurality of n-bit parent chromosomes are generated and assigned a cost based on evaluation of a cost function. Chromosomes with lower costs are selected for generating children. Child chromosomes are generated through a process of crossover and mutation of parent chromosomes to produce new child chromosomes. Child chromosomes with lower costs replace members of the population with higher costs to assure evolutionary advance to an optimal solution.

### SUMMARY

Systems and methods for selecting a value set associated with a set of parameters are disclosed. One embodiment of the present invention relates to a method. The method comprises determining real costs for a plurality of first value sets represented as a plurality of real chromosomes. Speculative costs are determined for a plurality of second value sets represented as a plurality of speculative chromosomes. The speculative chromosomes represent value set variations of the first value sets. Validation of speculative chromosomes is postponed by generating subsequent generations of speculative chromosomes and associated speculative costs from parents selected from at least one of the plurality of real chromosomes and the plurality of speculative chromosomes, until a predetermined validation criteria has been satisfied.

Another embodiment related to computer readable medium having computer executable instructions for performing a method. The method comprises executing a real cost function on a plurality of first value sets represented as a plurality of real chromosomes to generate a plurality of real costs for each of the plurality of real chromosomes. A genetic algorithm is executed to generate a plurality of speculative chromosomes. The speculative chromosomes represent value set variations of the first value sets. An incremental cost function is executed on the plurality of speculative chromosomes to generate a plurality of speculative costs for each of the plurality of speculative chromosomes. Execution of the genetic algorithm is repeated to produce subsequent generations of speculative chromosomes and execution of the incremental cost function is repeated on subsequent generations to provide speculative costs for the subsequent generations of speculative chromosomes, until a predetermined validation criteria has been satisfied.

In yet another embodiment, a system is disclosed. The system comprises a real cost function that generates real costs for each of a plurality of value sets represented as a plurality of real chromosomes. An incremental cost function generates a plurality of speculative costs corresponding to a plurality of value set variations of at least one of the plurality of real chromosomes. The plurality of value set variations represent a plurality of speculative chromosomes. A validator initiates a validation on at least one speculative chromosome upon satisfaction of a predetermined validation criteria. A validation comprises executing the real cost function on the at least one speculative chromosome to generate a real cost associated with at least one speculative chromosome.

### BRIEF DESCRIPTION OF THE DRAWINGS

FIG. 1 illustrates a block diagram of an embodiment of a system for selecting a value set associated with a set of parameters.

FIG. 2 is an embodiment of a graph that illustrates speculative generation count as a predetermined validation criteria.

FIG. 3 is an embodiment of a graph that illustrates a cost change limit associated with an incremental cost function as a predetermined validation criteria.

FIG. 4 is an embodiment of a graph that illustrates a cost change limits between speculative generations as a predetermined validation criteria.

FIG. 5 is an embodiment of a graph that illustrates one or more predetermined criteria for validation.

FIG. 6 illustrates a block diagram of an embodiment of a system for optimizing a circuit design.

FIG. 7 is a flow diagram that illustrates an embodiment of a methodology for selecting a value set associated with a set of parameters.

FIG. 8 is a flow diagram that illustrates another embodiment of a methodology for selecting a value set associated with a set of parameters.

FIG. 9 illustrates an embodiment of a computer system.

### DETAILED DESCRIPTION

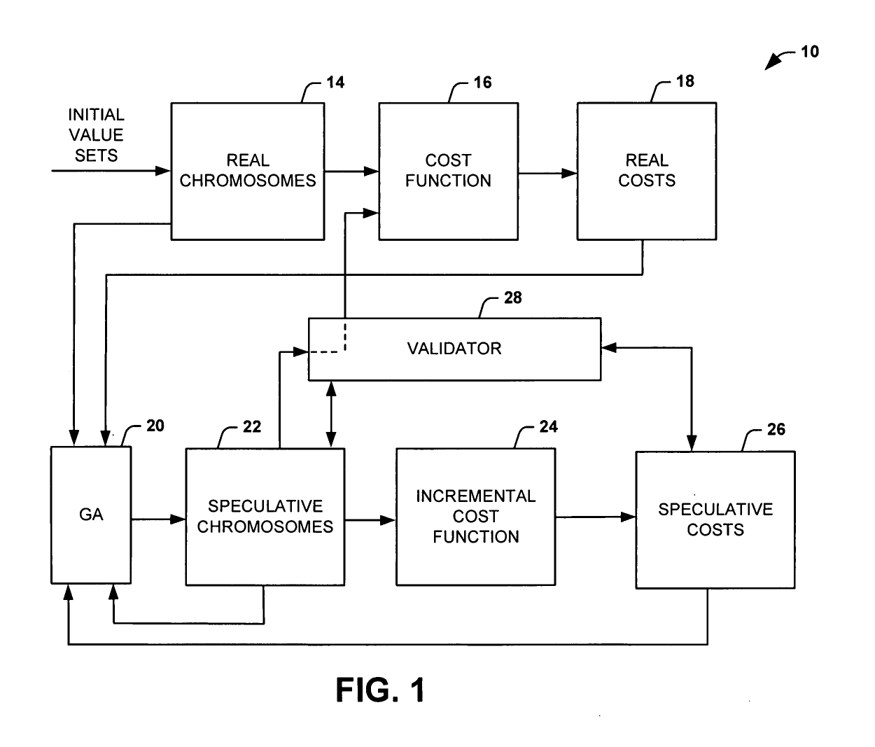

FIG. 1 illustrates a system 10 for selecting a value set associated with a set of parameters. The system 10 can be a computer, a server or some other computer readable medium that can execute computer readable instructions. For example, the components of the system 10 can be computer executable components, such as can be stored in a desired storage medium (e.g., random access memory, a hard disk drive, CD ROM, and the like), computer executable components running on a computer or design tool. The set of parameters can define properties or attributes associated with an optimizable function or structure.

An optimizable function or structure refers to a solution that can be improved with adjustment of values associated with one or more parameters to achieve a desirable acceptable solution. The optimizable function or structure can be, for example, a circuit design, a mathematical problem or some other optimizable function or structure. Each value set associated with the set of parameters represents a potential solution to the optimizable function or structure. The system 10 selects a value set based on a desired fitness value or desired minimal cost. A change in value in any one of the parameters defines a new value set. Each value set is represented by a chromosome, with each parameter representing a gene in the chromosome.

The terms “real” and “speculative” are used herein to distinguish the terms modified thereby. For example, a real cost function is a basis cost function that generates a cost (e.g., real cost) associated with a value set. A speculative cost function provides a cost (e.g., speculative cost) that is an approximate of the cost (e.g., real cost) that would be generated by the basis cost function. A speculative cost function can be arbitrary or a predetermined cost function that can be generated based on a real cost function value. The employment of a speculative cost function facilitates convergence of a desired solution by trading speed for accuracy.

A real chromosome represents a value set employed by a real cost function 16 (e.g., multi-variable cost function) to generate a real cost for a given value set. An initial set of real chromosomes 14 are provided to the real cost function 16 to generate real costs 18 associated with each of the real chromosomes 14.

The real chromosomes 14 and real costs 18 are employed by a genetic algorithm 20 to generate children chromosomes associated with parent chromosomes selected from the real chromosomes 14. The children chromosomes are generated through a process of crossover and mutation of parent chromosomes. The children chromosomes generated by the genetic algorithm 20 are referred to as speculative chromosomes 22. The children chromosomes derived from parents of the real chromosomes 14 are a first generation of speculative chromosomes. A speculative chromosome is a value set employed by an incremental cost function 24 to generate speculative or approximate costs 26 associated with value set variations of real chromosomes 14. The incremental cost function 24 provides an approximate or speculative cost for a given value set represented as a speculative chromosome. This enables an increase in speed of the system 10 since computing speculative costs, based on an approximation of the real costs, tends to be faster than computing the real costs employing the real cost function 14.

The genetic algorithm 20 can generate one or more generations of speculative chromosomes 22 based on selecting parent chromosomes from the speculative chromosomes 22. Alternatively, parents can be selected from the speculative chromosomes 22 and the real chromosomes 14, such that one parent is selected from the speculative chromosomes 22 and another parent is selected from the real chromosomes 14 for a given child chromosome. Speculative costs 26 can be approximated for speculative chromosomes in subsequent generations, via the incremental cost function 24, similar to the approximation performed for first generation speculative chromosomes.

The incremental cost function 24 approximates the cost effects of an incremental change in a value set between a parent chromosome and a child chromosome, and subtracts the cost effects from the cost determined for the parent chromosome to provide an approximate cost for the child chromosome. The speculative costs 26 can be approximated for one or more first generation speculative children chromosomes in a similar manner.

The genetic algorithm 20 can generate a second generation of speculative children from the first generation speculative chromosomes, which become speculative parents of the second generation. The second generation speculative parents can be selected based on minimum costs associated with the plurality of first generation speculative chromosomes. The success of genetic algorithms is dependent on generating costs representing improvements on the optimizable structure/problem through the process.

The incremental cost function 24 employs the second generation speculative parents selected from the first generation speculative chromosomes, the second generation speculative child chromosome generated from the second generation speculative parents, and the cost-evaluation of the second generation speculative parents to approximate a speculative cost for the second generation speculative child chromosome. This is repeated for each speculative child chromosome of the second generation. The genetic algorithm 20 can then generate a third generation of speculative chromosomes from parents selected from the second generation speculative chromosomes, and the incremental cost function 24 can determine speculative costs associated with the third generation speculative chromosomes. The third generation parents can be selected based on minimum costs associated with the plurality of second generation speculative chromosomes. This process can be repeated for subsequent generations, until it is decided that validation of the speculative chromosomes is desired.

A validator 28 monitors a predetermined validation criteria associated with the generating of speculative chromosomes and speculative costs to determine when to initiate a validation. Validation of the speculative chromosomes can be accomplished by invoking the execution of the real cost function 16 on the speculative chromosomes 22 to generate real costs 18 associated with the speculative chromosomes 22. The speculative chromosomes 22 then become real chromosomes with associated real costs. The validator 28 provides for postponing of validation until a predetermined validation criteria has been satisfied. This allows for several generations of speculative chromosomes and speculative costs to be generated before it is necessary to execute the real cost function 16, thus providing a gain in performance without undue inaccuracy.

The predetermined validation criteria can be based on a speculation count that corresponds to the number of speculative generations generated. Alternatively, the predetermined criteria can be based on a change in an incremental cost associated with a speculative chromosome generation, or a change in incremental cost associated with an incremental cost function. The predetermined validation criteria can be based on speculative costs converging (e.g., not changing significantly). Furthermore, predetermined validation criteria can be based on a predetermined error level being exceeded. It is to be appreciated that the inherent error of the incremental cost function 24 may increase with each generation of speculative chromosomes, since approximate cost might be based on previous approximations.

Once the predetermined validation criteria has been satisfied, the validator 28 provides the speculative chromosomes 22 to the real cost function 16 to generate real costs 18 associated with the speculative chromosomes 22. The speculative chromosomes 22 then become real chromosomes 14. The new real chromosomes are evaluated to determine if a desirable solution has been satisfied. The desirable solution can be based on achieving a minimum cost associated with a real chromosome or when real costs converge. If the desirable solution has not been satisfied, a new incremental cost function can be generated based on a new set of real chromosomes and real costs. The process of generating new generations of speculative chromosomes via the genetic algorithm 20 and determining speculative costs based on the new incremental cost function can be repeated, until the validator 28 initiates a validation based on the same or different predetermined validation criteria being satisfied. This process repeats until a desirable solution or value set based on the real cost function 16 is satisfied.

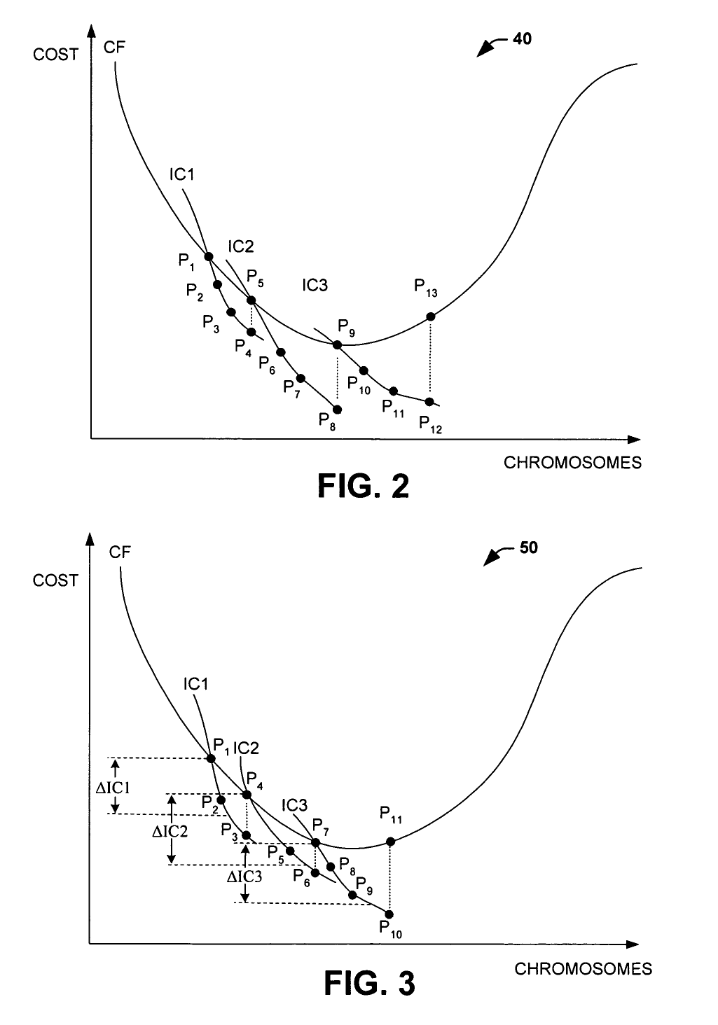

FIG. 2 is a graph 40 that illustrates a relationship between an exemplary real cost function (CF) and a plurality of incremental cost functions (IC1-IC3), which employs speculative generation count as a predetermined validation criteria. Speculative generation count refers to a degree of speculation assigned to a speculative chromosome. For example, a speculative chromosome generated by real parent chromosomes have a speculative count of one, while speculative chromosomes generated by speculative parent chromosomes with a speculative count of one have a speculative count of two. Speculative chromosomes have a speculation count that is one greater than the highest speculative count of the parent chromosomes.

The graph 40 illustrates the real cost function (CF) and the plurality of incremental cost functions (IC1-IC3) in two dimensions. However, it is to be appreciated that a multi-variable cost function will have as many dimensions as variables or parameters in the cost function. For example, a k variable cost function has k dimensions, were k is an integer greater than one. The number of variables and associated dimensions map to a single cost value. The graph 40 illustrates costs versus chromosome sets.

As illustrated in the graph 40, a real pool P1, corresponding to a pool of real chromosomes and an associated real cost resides on the cost function CF. The real pool P1 is assigned a real cost based on the minimum cost chromosome associated with the real pool P1. This allows mapping of an entire pool to a single cost. A first incremental cost function (IC1) is generated beginning with the real pool P1 and its assigned real cost. An incremental cost function can be arbitrary or a predetermined cost function that can be generated based on a real cost function value.

A genetic algorithm employs one or more real chromosomes as parents selected from the real pool P1 to generate a first generation of speculative chromosomes. The incremental cost function IC1 provides associated speculative costs to the set of speculative chromosomes. The first generation of speculative chromosomes and associated speculative costs are stored in a speculative pool P2. The first generation speculative chromosomes are assigned a speculative generation count of one. The speculative pool P2 is assigned a speculative cost based on the minimum cost chromosome associated with the speculative pool P2.

A genetic algorithm employs one or more speculative chromosomes as parents selected from the speculative pool P2 to generate a second generation of speculative children chromosomes. Alternatively, parents can be selected from the real pool P1 and the speculative pool P2. The incremental cost function IC1 provides associated speculative costs to the set of speculative children chromosomes. The second generation speculative chromosomes are assigned a speculative generation count of two. A new speculative pool P3 is formed that can be a combination of speculative chromosomes from the first generation and the second generation of speculative chromosomes with higher cost speculative chromosomes being discarded. The speculative pool P3 is assigned a speculative cost based on the minimum cost chromosome associated with the speculative pool P3.

A genetic algorithm employs one or more speculative chromosomes as parents selected from the speculative pool P3 to generate third generation of speculative children chromosomes. Alternatively, parents can be selected from the real pool P1 and the speculative pools P2 and P3. The incremental cost function IC1 provides associated speculative costs to the third generation of speculative children chromosomes. The third generation speculative chromosomes are assigned a speculative generation count of three. A new speculative pool P4 is formed that can be a combination of speculative chromosomes from the first generation, the second generation and the third generation of speculative chromosomes with higher cost speculative chromosomes being discarded. The speculative pool P4 is assigned a speculative cost based on the minimum cost chromosome associated with the speculative pool P4.

A validation is initiated on the speculative chromosomes residing in the speculative pool P4, since the speculative pool includes at least one speculative chromosome with a speculative count of three. Alternatively, only the speculative chromosomes with a count of three can be validated. It is to be appreciated that the speculative count of three is employed for illustrative purposes as more or less than a speculative count of three can be employed as validation criteria to determine whether or not to validate for a given incremental cost function.

Once the validation is initiated, one or more speculative chromosomes of the speculative pool P4 are provided to the real cost function CF. A new set of real chromosomes and real costs are generated. The new set of real chromosomes and real costs are combined with the real pool P1 with lower cost chromosomes replacing higher cost chromosomes, such that a new real pool P5 is generated, and moved to the real cost function CF.

A second incremental cost function (IC2) is generated beginning with the real pool P5 and its assigned real cost. A genetic algorithm employs one or more real chromosomes as parents selected from the real pool P5 to generate first generation of speculative chromosomes. The incremental cost function IC2 provides associated speculative costs to the set of speculative chromosomes. The first generation speculative chromosomes are assigned a speculative generation count of one. The set of speculative chromosomes and associated speculative costs are stored in a speculative pool P6. The speculative pool P6 is assigned a speculative cost based on the minimum cost chromosome associated with the speculative pool P6.

The speculative pool P6 is employed to generate a new generation of speculative chromosomes and associated speculative costs. Alternatively, parents can be selected from the real pool P5 and the speculative pool P6. A genetic algorithm generates a second generation of speculative children chromosomes from the selected parents. The incremental cost function IC2 provides associated speculative costs to the second generation speculative children chromosomes. The second generation speculative chromosomes are assigned a speculative generation count of two. A new speculative pool P7 is formed that can be a combination of speculative chromosomes from the first generation and the second generation of speculative chromosomes with higher cost speculative chromosomes being discarded. The speculative pool P7 is assigned a speculative cost based on the minimum cost chromosome associated with the speculative pool P7.

The speculative pool P7 is employed to generate a third generation of speculative chromosomes and associated speculative costs. Alternatively, parents can be selected from the real pool P5 and the speculative pools P6 and P7. A genetic algorithm employs the selected parents to generate a third generation of speculative children chromosomes. The third generation speculative chromosomes are assigned a speculative generation count of three. A new speculative pool P8 is formed that can be a combination of speculative chromosomes from the first generation, the second generation and the third generation of speculative chromosomes with higher cost speculative chromosomes being discarded. The speculative pool P8 is assigned a speculative cost based on the minimum cost chromosome associated with the speculative pool P8.

A validation is initiated on the speculative chromosomes residing in the speculative pool P8, since the speculative pool P8 includes at least one speculative chromosome with a speculative count of three. It is to be appreciated that the speculative count of three is employed for illustrative purposes and more or less than a speculative count of three can be employed to determine whether or not to validate for a given incremental cost function. Additionally, a speculative count that initiates a validation can differ between cost functions.

Upon validation, one or more speculative chromosomes of the speculative pool P8 are provided to the real cost function (CF). A new set of real chromosomes and real costs are generated. The new set of real chromosomes and real costs are combined with the real pool P5 with lower cost chromosomes replacing higher cost chromosomes, such that a new real pool P9 is generated.

The set of real chromosomes in the pool P9 are employed to generate a third incremental cost function (IC3). The set of real chromosomes in real pool P9 and associated costs are employed to generate first generation of speculative chromosomes and associated speculative costs stored in a speculative pool P10.

The speculative pool P10 is employed to generate a second generation of speculative chromosomes and associated speculative costs. Alternatively, parents can be selected from the real pool P9 and the speculative pool P10. A genetic algorithm generates a set of speculative children chromosomes from the selected parents. The incremental cost function IC3 provides associated speculative costs to the set of speculative children chromosomes.

The speculative pool P10 is employed to generate a second generation of speculative chromosomes and associated speculative costs. Alternatively, parents can be selected from the real pool P9 and the speculative pool P10. A genetic algorithm employs the selected parents to generate a second generation of speculative children chromosomes. The incremental cost function IC3 provides associated speculative costs to the new generation of speculative children chromosomes. The second generation speculative chromosomes are assigned a speculative generation count of two. A new speculative pool P11 is formed that can be a combination of speculative chromosomes from the first generation and the second generation of speculative chromosomes with higher cost speculative chromosomes being discarded. The speculative pool P11 is assigned a speculative cost based on the minimum cost chromosome associated with the speculative pool P11.

The speculative pool P11 is employed to generate a third generation of speculative chromosomes and associated speculative costs. Alternatively, parents can be selected from the real pool P9 and the speculative pools P10 and P11. A genetic algorithm employs the selected parents to generate a third generation of speculative children chromosomes. The third generation speculative chromosomes are assigned a speculative generation count of three. A new speculative pool P12 is formed that can be a combination of speculative chromosomes from the first generation, the second generation and/or the third generation of speculative chromosomes with higher cost speculative chromosomes being discarded. The speculative pool P12 is assigned a speculative cost based on the minimum cost chromosome associated with the speculative pool P12.

A validation is initiated on the speculative chromosomes residing in the speculative pool P12, since the speculative pool P12 includes at least one speculative chromosome with a speculative count of three. Upon validation, one or more speculative chromosomes of the speculative pool P12 are provided to the real cost function (CF). A new set of real chromosomes and real costs are generated. The new set of real chromosomes and real costs are combined with the real pool P9 with lower cost chromosomes replacing higher cost chromosomes, such that a new real pool P13 is generated.

In the example of FIG. 2, It is determined that the minimal cost assigned to the real pool P13 is higher than the minimal cost assigned to the real pool P9. Therefore, the real pool P9 offers a better solution than P13. A minimal cost real chromosome can be selected from the real chromosomes represented at P9 as a desirable solution. The selection routine then terminates. It is to be appreciated that more or less than three incremental cost functions can be generated to determine a desirable solution associated with the real cost function (CF).

FIG. 3 is a graph 50 that illustrates a relationship between an exemplary real cost function (CF) and a plurality of incremental cost functions (IC1-IC3), which employs a cost change limit associated with an incremental cost function as a predetermined criteria for validation. A cost change limit associated with an incremental cost function relates to a cost change between a real pool and a speculative pool of a given speculative generation for a given incremental cost function. A cost difference between a speculative generation and the real pool is compared with a predetermined cost change limit for each speculative generation. The cost change limit can be determined based on an associated incremental cost function, can be arbitrary or a matter of design choice. The graph 50 illustrates the real cost function (CF) and the plurality of incremental cost functions (IC1-IC3) in two dimensions. However, it is to be appreciated that a multi-variable cost function will have as many dimensions as variables or parameters in the cost function. The number of variables and associated dimensions map to a single cost value. The graph 50 illustrates costs versus chromosomes sets.

As illustrated in the graph 50, a first real pool P1, corresponding to a pool of real chromosomes and an associated real cost resides on the cost function CF. A first incremental cost function (IC1) is generated beginning with the real pool P1 and its assigned real cost. A genetic algorithm employs one or more real chromosomes as parents selected from the real pool P1 to generate first generation of speculative chromosomes. The incremental cost function IC1 provides associated speculative costs to the first generation of speculative chromosomes. The set of speculative chromosomes and associated speculative costs are stored in a speculative pool P2. The speculative pool P2 is assigned a speculative cost based on the minimum cost chromosome associated with the speculative pool P2. The speculative cost change between the real pool P1 and the speculative pool P2 is less than a speculative cost change limit ΔIC1 associated with the incremental cost function IC1.

Therefore, the speculative pool P2 is employed to generate a new generation of speculative chromosomes and associated speculative costs. A genetic algorithm employs one or more speculative chromosomes as parents selected from the speculative pool P2 to generate a second generation of speculative children chromosomes. Alternatively, parents can be selected from the real pool P1 and the speculative pool P2. The incremental cost function IC1 provides associated speculative costs to the set of speculative children chromosomes. The speculative children chromosomes replace speculative parent chromosomes with higher costs, such that a new speculative pool P3 is formed with a lower cost. The speculative pool P3 is assigned a speculative cost based on the minimum cost chromosome associated with the speculative pool P3. The speculative cost change between the real pool P1 and the speculative pool P3 is greater than the speculative cost change limit ΔIC1 associated with the incremental cost function IC1. Therefore, a validation of at least one speculative chromosome in the speculative pool P3 is initiated.

Once the validation is initiated, one or more speculative chromosomes of the speculative pool P3 are provided to the real cost function CF. A new set of real chromosomes and associated real costs are generated. The new set of real chromosomes and real costs are combined with the real pool P1 with lower cost chromosomes replacing higher cost chromosomes, such that a new real pool P4 is generated, and moved to the real cost function CF.

A second incremental cost function (IC2) is generated beginning with the real pool P4 and its assigned real cost. A genetic algorithm employs one or more real chromosomes as parents selected from the real pool P4 to generate a first generation of speculative chromosomes. The incremental cost function IC2 provides associated speculative costs to the first generation of speculative chromosomes. The set of speculative chromosomes and associated speculative costs are stored in a speculative pool P5. The speculative pool P5 is assigned a speculative cost based on the minimum cost chromosome associated with the speculative pool P5. The speculative cost change between the real pool P4 and the speculative pool P5 is less than a speculative cost change limit ΔIC2 associated with the incremental cost function IC2.

The speculative pool P5 is employed to generate a second generation of speculative chromosomes and associated speculative costs. Alternatively, parents can be selected from the real pool P4 and the speculative pool P5. A genetic algorithm generates a set of speculative children chromosomes from the selected parents. The incremental cost function IC2 provides associated speculative costs to the second generation of speculative children chromosomes. The speculative children chromosomes replace speculative parent chromosomes with higher costs, such that a new speculative pool P6 is formed with a lower cost. The speculative pool P6 is assigned a speculative cost based on the minimum cost chromosome associated with the speculative pool P6. The speculative cost change between the real pool P4 and the speculative pool P6 is greater than the speculative cost change limit ΔIC2 associated with the incremental cost function IC2. Therefore, a validation of at least one speculative chromosome in the speculative pool P6 is initiated.

Upon validation, one or more speculative chromosomes of the speculative pool P6 are provided to the real cost function (CF). A new set of real chromosomes and real costs are generated. The new set of real chromosomes and real costs are combined with the real pool P4 with lower cost chromosomes replacing higher cost chromosomes, such that a new real pool P7 is generated.

The set of real chromosomes in the real pool P7 are employed to generate a third incremental cost function (IC3). The set of real chromosomes in real pool P7 and associated costs are employed to generate a first generation of speculative chromosomes and associated speculative costs stored in a speculative pool P8. The speculative cost change between the real pool P7 and the speculative pool P8 is less than a speculative cost change limit ΔIC3 associated with the incremental cost function IC3. Therefore, validation is postponed and a new generation of speculative chromosomes is generated.

The speculative pool P8 is employed to generate a second generation of speculative chromosomes and associated speculative costs. Alternatively, parents can be selected from the real pool P7 and the speculative pool P8. A genetic algorithm generates a set of speculative children chromosomes from the selected parents. The incremental cost function IC3 provides associated speculative costs to the set of speculative children chromosomes. The speculative children chromosomes replace speculative parent chromosomes with higher costs, such that a new speculative pool P9 is formed with a lower cost. The speculative pool P9 is assigned a speculative cost based on the minimum cost chromosome associated with the speculative pool P9. The speculative cost change between the real pool P7 and the speculative pool P9 is less than a speculative cost change limit ΔIC3 associated with the incremental cost function IC3. Therefore, validation is postponed and a new generation of speculative chromosomes is generated.

The speculative pool P9 is employed to generate a third generation of speculative chromosomes and associated speculative costs. Alternatively, parents can be selected from the real pool P7 and the speculative pools P8 and P9. A genetic algorithm employs the selected parents to generate a third generation of speculative children chromosomes. The incremental cost function IC3 provides associated speculative costs to the third generation of speculative children chromosomes. The speculative children chromosomes replace speculative parent chromosomes with higher costs, such that a new speculative pool P10 is formed with a lower cost. The speculative cost change between the real pool P7 and the speculative pool P10 is greater than the speculative cost change limit ΔIC3 associated with the incremental cost function IC3. Therefore, a validation of at least one speculative chromosome in the speculative pool P10 is initiated.

Upon validation, one or more speculative chromosomes of the speculative pool P10 are provided to the real cost function (CF). A new set of real chromosomes and real costs are generated. The new set of real chromosomes and real costs are combined with the real pool P7 with lower cost chromosomes replacing higher cost chromosomes, such that a new real pool P11 is generated.

It is determined that the minimal cost assigned to the real pool P11 is about the same as the minimal cost assigned to the real pool P7. Therefore, a solution can be selected from either the real pool P7 or the real P11. The selection routine then terminates. It is to be appreciated that more or less than three incremental cost functions can be generated to determine a desirable solution associated with the real cost function (CF).

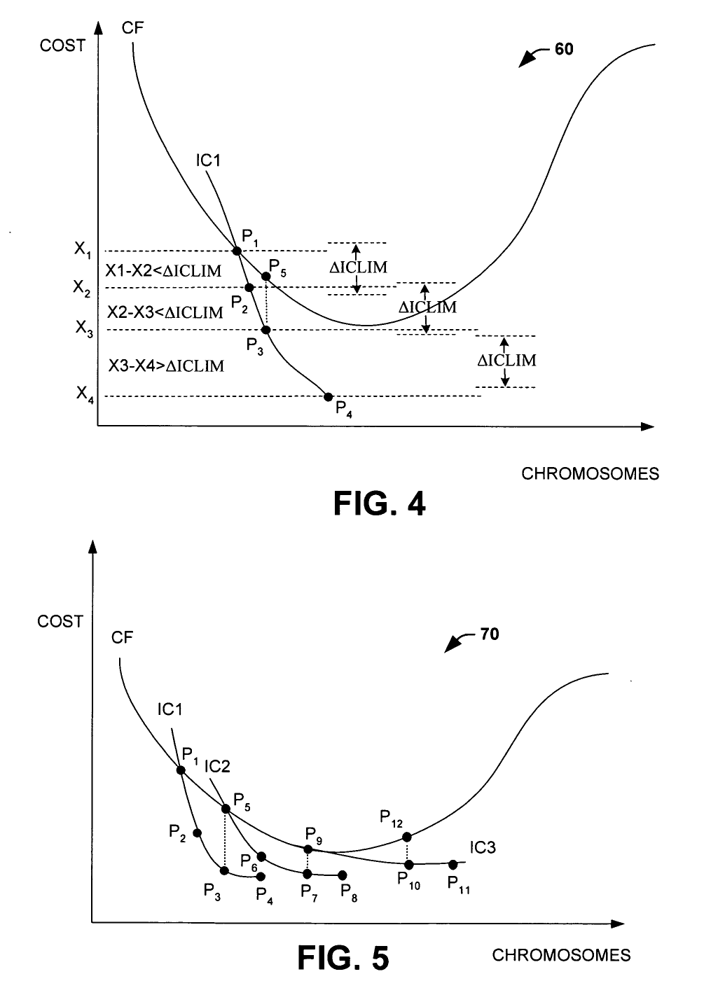

FIG. 4 is a graph 60 that illustrates a relationship between an exemplary real cost function (CF) and an incremental cost function, which employs a cost change limit (ΔICLIM) between speculative generations as a predetermined criteria for validation. A cost change limit between speculative generations relates to a cost change between a given speculative generation and its subsequent speculative generation for a given incremental cost function. A cost difference between a speculative generation and its subsequent generation is compared with a predetermined cost change limit. The cost change limit can be determined based on an associated incremental cost function, can be arbitrary or a matter of design choice. The graph 60 illustrates the real cost function (CF) and the incremental cost function in two dimensions. However, it is to be appreciated that a multi-variable cost function will have as many dimensions as variables or parameters in the cost function.

As illustrated in the graph 60, a first real pool P1, corresponding to a pool of real chromosomes and an associated real cost resides on the cost function CF. A first incremental cost function (IC1) is generated beginning with the real pool P1 and its assigned real cost. A genetic algorithm employs one or more real chromosomes as parents selected from the real pool P1 to generate a first generation of speculative chromosomes. The incremental cost function IC1 provides associated speculative costs to the set of speculative chromosomes. The set of speculative chromosomes and associated speculative costs are stored in a speculative pool P2. The speculative pool P2 is assigned a speculative cost based on the minimum cost chromosome associated with the speculative pool P2. The speculative cost difference (X₁−X₂) between the real pool P1 and the speculative pool P2 is determined to be less than a speculative cost change limit ΔICLIM associated with the incremental cost function IC1.

Since X₁−X₂<ΔICLIM, the speculative pool P2 is employed to generate a second generation of speculative chromosomes and associated speculative costs. A genetic algorithm employs one or more speculative chromosomes as parents selected from the speculative pool P2 to generate a second generation of speculative children chromosomes. Alternatively, parents can be selected from the real pool P1 and the speculative pool P2. The incremental cost function IC1 provides associated speculative costs to the set of speculative children chromosomes. The speculative children chromosomes replace speculative parent chromosomes with higher costs, such that a new speculative pool P3 is formed with a lower cost. The speculative pool P3 is assigned a speculative cost based on the minimum cost chromosome associated with the speculative pool P3. The speculative cost change (X₂−X₃) between the speculative pool P3 and the speculative pool P2 is determined to be less than the speculative cost change limit ΔICLIM.

Since X₂−X₃<ΔICLIM, the speculative pool P3 is employed to generate a third generation of speculative chromosomes and associated speculative costs. A genetic algorithm employs one or more speculative chromosomes as parents selected from the speculative pool P3 to generate a set of speculative children chromosomes. Alternatively, parents can be selected from the real pool P1 and the speculative pools P2 and P3. The incremental cost function IC1 provides associated speculative costs to the third generation of speculative children chromosomes. The speculative children chromosomes replace speculative parent chromosomes with higher costs, such that a new speculative pool P4 is formed with a lower cost. The speculative pool P4 is assigned a speculative cost based on the minimum cost chromosome associated with the speculative pool P4. The speculative cost change between the speculative pool P4 and the speculative pool P3 is determined to be greater than the speculative cost change limit ΔICLIM. Therefore, a validation of at least one speculative chromosome in the speculative pool P4 is initiated.

FIG. 5 is a graph 70 that illustrates a relationship between an exemplary real cost function (CF) and a plurality of incremental cost functions (IC1-IC3), which employ speculative cost convergence as a predetermined criteria for validation. Speculative cost convergence relates to speculative costs converging to a solution due to insubstantial cost changes for additional speculative generations of chromosomes. Although the examples in the graph are illustrated with respect to convergence occurring after an insubstantial change in speculative costs between a single generation, it is to be appreciated that convergence can occur after several generations without a substantial change in speculative costs. The graph 70 illustrates the real cost function (CF) and the plurality of incremental cost functions (IC1-IC3) in two dimensions. However, it is to be appreciated that a multi-variable cost function will have as many dimensions as variables or parameters in the cost function.

As illustrated in the graph 70, a first real pool P1, corresponding to a pool of real chromosomes and an associated real cost resides on the cost function CF. A first incremental cost function (IC1) is generated beginning with the real pool P1 and its assigned real cost. A genetic algorithm employs one or more real chromosomes as parents selected from the real pool P1 to generate a first generation of speculative chromosomes. The incremental cost function IC1 provides associated speculative costs to the first generation of speculative chromosomes. The first generation of speculative chromosomes and associated speculative costs are stored in a speculative pool P2. The speculative pool P2 is assigned a speculative cost based on the minimum cost chromosome associated with the speculative pool P2.

The speculative pool P2 is employed to generate a new generation of speculative chromosomes via a genetic algorithm. The incremental cost function IC1 provides associated speculative costs to the set of speculative children chromosomes associated with a new speculative pool P3, which is assigned a speculative cost based on the minimum cost chromosome associated with the speculative pool P3. The speculative pool P3 is employed to generate a new generation of speculative chromosomes via a genetic algorithm. The incremental cost function IC1 provides associated speculative costs to the set of speculative children chromosomes associated with a new speculative pool P4, which is assigned a speculative cost based on the minimum cost chromosome associated with the speculative pool P4. However, it is determined that the speculative cost associated with the speculative pool P4 has not substantially changed with respect to the speculative cost associated with the speculative pool P3. The speculative costs have converged and a validation is initiated on at least one chromosome associated with the speculative pool P3.

Once the validation is initiated, one or more speculative chromosomes of the speculative pool P3 are provided to the real cost function CF. A new set of real chromosomes and real costs are generated to provide a pool P5. A second incremental cost function (IC2) is generated beginning with the real pool P5 and its assigned real cost. A genetic algorithm employs one or more real chromosomes as parents selected from the real pool P5 to generate a set of speculative chromosomes. The incremental cost function IC2 provides associated speculative costs to the set of speculative chromosomes. The set of speculative chromosomes and associated speculative costs are stored in a speculative pool P6. The speculative pool P6 is assigned a speculative cost based on the minimum cost chromosome associated with the speculative pool P6.

The speculative pool P6 is employed to generate a new generation of speculative chromosomes via a genetic algorithm. The incremental cost function IC2 provides associated speculative costs to the set of speculative children chromosomes to provide a new speculative pool P7. The speculative children chromosomes replace speculative parent chromosomes with higher costs, such that a new speculative pool P6 is formed with a lower cost. The speculative pool P7 is assigned a speculative cost based on the minimum cost chromosome associated with the speculative pool P7. The speculative pool P7 is employed to generate a new generation of speculative chromosomes via a genetic algorithm. The incremental cost function IC2 provides associated speculative costs to the set of speculative children chromosomes associated with a new speculative pool P8, which is assigned a speculative cost based on the minimum cost chromosome associated with the speculative pool P8. However, it is determined that the speculative cost associated with the speculative pool P8 has not significantly changed with respect to the speculative cost associated with the speculative pool P7. The speculative costs have converged and a validation is initiated on at least one chromosome associated with the speculative pool P7.

Upon validation, one or more speculative chromosomes of the speculative pool P7 are provided to the real cost function (CF). A new set of real chromosomes and real costs are generated. The new set of real chromosomes and real costs are combined with the real pool P5 with lower cost chromosomes replacing higher cost chromosomes, such that a new real pool P9 is generated.

The set of real chromosomes in the pool P9 are employed to generate a third incremental cost function (IC3). The set of real chromosomes in real pool P9 and associated costs are employed to generate a set of speculative chromosomes and associated speculative costs stored in a speculative pool P10. The speculative pool P10 is employed to generate a new generation of speculative chromosomes and associated speculative costs. The incremental cost function IC3 provides associated speculative costs to the set of speculative children chromosomes to provide a new speculative pool P11.

However, it is determined that the speculative cost associated with the speculative pool P11 has not significantly changed with respect to the speculative cost associated with the speculative pool P10. Validation is initiated on at least one speculative chromosome associated with the speculative pool P10.

Upon validation, one or more speculative chromosomes of the speculative pool P10 are provided to the real cost function (CF). A new set of real chromosomes and real costs are generated. The new set of real chromosomes and real costs generate a new real pool P12.

It is determined that the minimal cost assigned to the real pool P12 is greater than the minimal cost assigned to the real pool P9. A minimal cost real chromosome can be selected from the real pool P9 as a desirable solution. The selection routine then terminates. It is to be appreciated that more or less than three incremental cost functions can be generated to determine a desirable solution associated with the real cost function (CF).

FIGS. 2-5 illustrate several different predetermined validation criteria techniques that can be employed to postpone validation of speculative chromosomes and associated speculative costs. It is to be appreciated that several other predetermined criteria can be employed. For example, an execution time limit for generating new speculative generations and new speculative costs can be employed as a predetermined validation criteria. Additionally, errors can increase with each generation of speculation for a given cost function. Therefore, a predetermined validation criteria can be when a predetermined error level has been satisfied. Furthermore, a combination of criteria can be employed to determine when to initiate a validation.

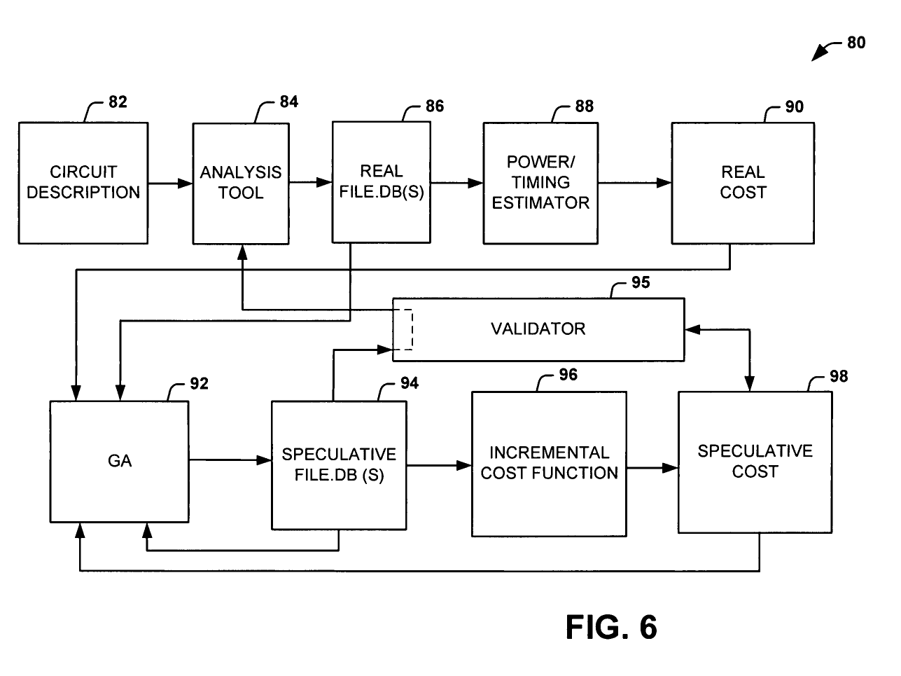

FIG. 6 illustrates a system 80 for optimizing a circuit design. The system 80 employs a circuit design description 82 to provide information to an analysis tool 84. The design description 82 can include transistor netlists, design netlists, design parasitic data and timing constraints associated with the circuit design. The analysis tool 84 executes a device modification and timing algorithm to optimize a circuit design. For example, the analysis tool 84 can be a static timing analysis tool (e.g., PATHMILL® by Synopsys) for block and chip timing verification. A static timing analysis tool will generate a plurality of circuit design configurations that correspond to device changes (e.g., transistor sizing, cell device modifications) based on timing and delay analysis to optimize the circuit design based on speed, power and area.

Alternatively, the analysis tool 84 can be a transistor autosizer (e.g., AMPS® by Synopsys). Most transistor autosizers rely on heuristic approaches that focus on finding the best combination that will meet user-defined power and speed goals without changing the functionality of the design. The transistor autosizers employ an original circuit design description to generate a plurality of circuit sizing configurations that define different optimized cell netlist configurations.

The analysis tool 84 performs timing analysis, transistor sizing optimization, device modifications and/or power analysis on the circuit design description 82. The analysis tool 84 executes timing analysis and modifies transistor sizes and/or circuit cell configurations to optimize the circuit design without disturbing the functionality associated with the circuit design. The analysis tool 84 generates one or more real file data bases 86 (File.DB(s)). Each of the one or more real file data bases 86 defines a circuit configuration, and a potential circuit design solution. Each circuit configuration or real file data base 86 is represented as a real chromosome. Any change in the circuit design parameter values (e.g., device width, device length, circuit types, cell types) defines a new real chromosome associated with the circuit design.

The information associated with the one or more real file data bases 86 is provided to a power/timing estimator 88 that generates real costs associated with each real file data base 86, as a function of power and timing characteristics. The analysis tool 84 and the power/timing estimator 88 cooperate to define a real cost function associated with optimization of the circuit design.

A genetic algorithm 92 generates a first generation of speculative chromosomes in the form of speculative file data bases 94 (File.DB(s)) through a process of crossover and mutation of parent chromosomes selected from the real file data bases 86 and associated real costs 90. The speculative file data bases 94 are provided to an incremental cost function 96, which determines speculative costs 98 associated with respective speculative file database 94.

The incremental cost function 96 employs the incremental difference between parent chromosomes having real file data bases 86 and speculative child chromosomes having speculative file data bases 94. The incremental difference and the real cost associated with the real parent file data bases 86 is employed to provide a speculative cost associated with each speculative child chromosome or speculative file data base 94. For example, a change in a circuit design parameter value, such as gate width can be made to generate a speculative file data base from one or more real file databases. An estimated change in power can be determined based on the gate width change. The estimated change in power and the power computed by the power timing/estimator 88 for the real file data base 86 can be employed to determine an approximate power associated with the speculative file data base 94.

Parent chromosomes are then selected from the speculative chromosomes 94, such that speculative chromosomes become parents of a second generation of speculative chromosomes. Alternatively, parents can be selected from the speculative chromosomes and real chromosomes, such that one parent is selected from the speculative file data base 94 and another parent is selected from the real file data base 86 for a given child chromosome. The genetic algorithm 92 produces a subsequent generation of speculative file data bases. The speculative and/or real parent chromosomes can be selected based on minimum speculative costs. Alternatively, multiple combinations of speculative parents and/or real parents can be selected to generate various second generation children chromosomes.

The second generation speculative file data bases 94 and associated speculative costs 98 are provided to the incremental cost function 96. The incremental cost function 96 employs the incremental difference of circuit design configuration associated with the first generation speculative file data bases and the second generation speculative file data bases. The incremental difference and the speculative cost associated with the first generation speculative chromosome parents are employed to provide a speculative cost associated with each second generation speculative child chromosomes. This process can then be repeated for subsequent generations (e.g., 3^{rd} generation, 4^{th} generation, etc.) of speculative file data bases employing parents of a previous generation.

A validator 95 monitors the generating of speculative chromosomes and speculative costs to determine when to initiate a validation. Validation of the speculative chromosomes is accomplished by invoking the execution of the real cost function (84, 88) on the speculative file data bases 94 to generate real costs 90 associated with the speculative chromosomes. The speculative file data bases 94 then become real file data bases 86 with associated real costs. The validator 95 provides for postponing of validation until a predetermined validation criteria has occurred. This allows for several generations of speculative chromosomes to be generated and associated speculative costs to be determined before it is necessary to execute the real cost function.

The predetermined criteria can be based on a speculation generation count that corresponds to the speculative generation associated with a given speculative chromosome. Alternatively, the predetermined criteria can be based a speculative cost difference between speculative chromosome generations exceeding a predetermined cost change limit, or can be based on a speculative cost difference between a real chromosome and a speculative chromosome generations change exceeding a predetermined cost change limit. The predetermined validation criteria can be based on speculative costs converging (e.g., not changing significantly). Furthermore, predetermined validation criteria can be based on a predetermined error level being exceeded.

Once the predetermined criteria has been satisfied, the validator 95 invokes validation by halting execution of the genetic algorithm 92 and the incremental cost function 96. The validator 95 then provides the speculative filed data bases 94 to the real cost function (84, 88) to generate real costs 90 associated with the speculative chromosomes. The speculative chromosomes then become real chromosomes. The new real chromosomes are evaluated to determine if a desirable solution has been satisfied. The desirable solution can be based on achieving a minimum cost associated with a real chromosome or when real costs converge. If the desirable solution has not been satisfied, a new incremental cost function can be generated based on a new set of real chromosomes and real costs. The process of generating new generations of speculative chromosomes via the genetic algorithm 02 and speculative costs based on the new incremental cost function can be repeated, until the validator 95 initiates a validation of the new speculative chromosome generations. This process repeats until a desirable solution or value set based on the real cost function has been satisfied.

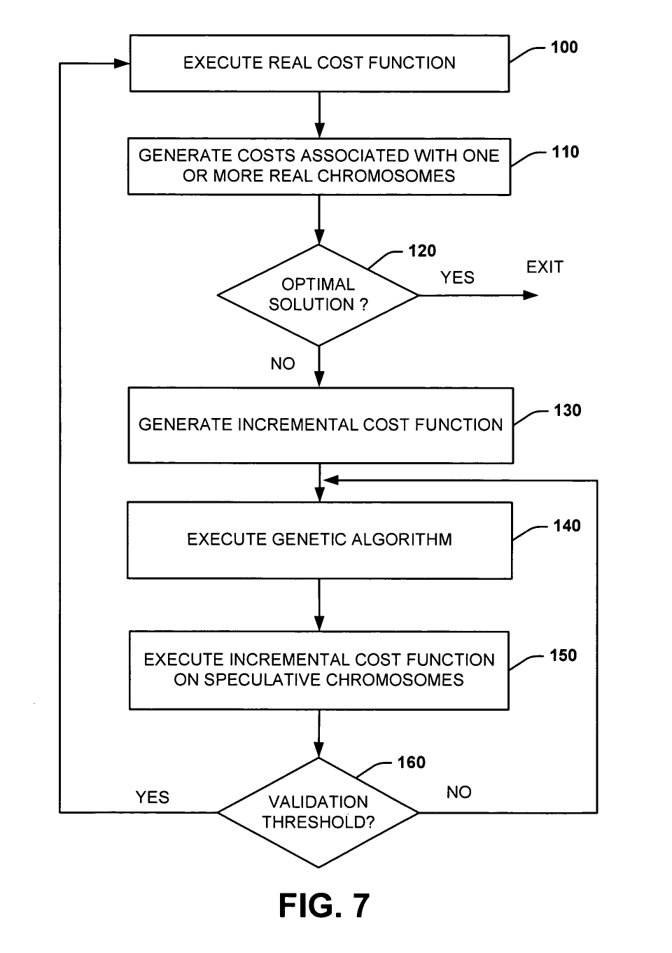

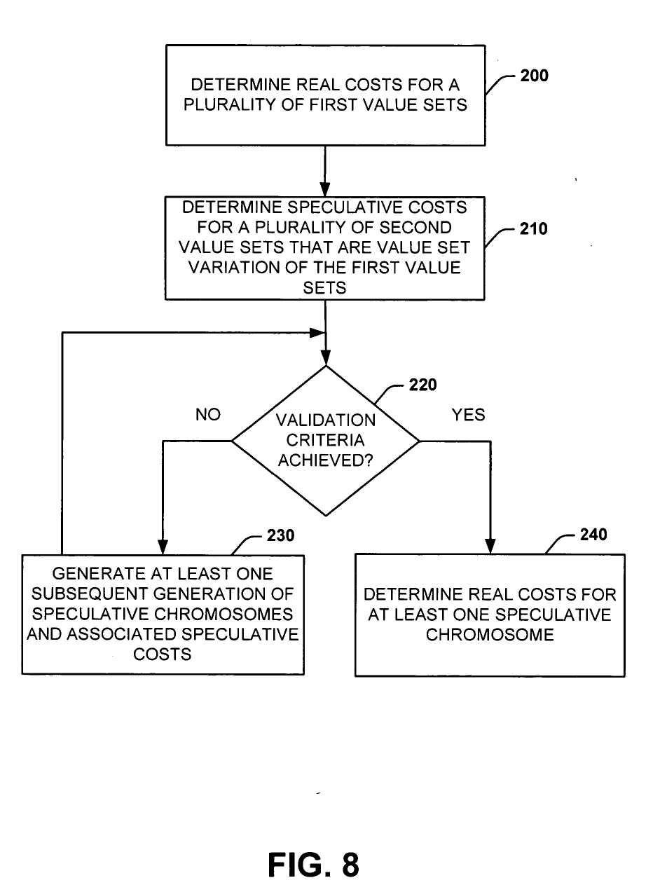

In view of the foregoing structural and functional features described above, certain methodologies that can be implemented will be better appreciated with reference to FIGS. 7-8. While, for purposes of simplicity of explanation, the methodologies of FIGS. 7-8 are shown and described as being implemented serially, it is to be understood and appreciated that the illustrated actions, in other embodiments, may occur in different orders and/or concurrently with other actions. Moreover, not all illustrated features may be required to implement a methodology.

It is to be further understood that the following methodology can be implemented in hardware, software, or any combination thereof. For example, in one embodiment the methodologies can be implemented as computer executable instructions, such as can be stored in a desired storage medium (e.g., random access memory, a hard disk drive, CD ROM, and the like). In another embodiment, a methodology can be implemented as computer executable instructions running on a computer or design tool.

FIG. 7 illustrates a methodology for optimizing a value set associated with a set of parameters. At 100, a real cost function is executed on one or more value sets associated with a set of parameters. Each value set represents a real chromosome with each parameter value representing a gene associated with the real chromosome. For example, the set of parameters can be parameters (e.g., device width, device length, circuit types, cell types) associated with a circuit design. At 110, a real cost function generates real costs for each of the one or more real chromosomes that represent one or more value sets associated with a set of parameters. The real chromosomes and real costs can be stored in a real pool. The real chromosomes and real costs can be sorted based on minimum real costs associated with a given chromosome. At 120, it is determined if a desirable solution has been obtained by analyzing the costs associated with the real chromosomes in the real pool. If a desirable solution has been satisfied (YES), the methodology terminates or exits. If a desirable solution has not been satisfied (NO), the methodology proceeds to 130.

At 130, an incremental cost function is generated based on the real chromosomes in the real pool and an associated minimum real cost assigned to the real pool. The minimum real cost assigned to the real pool can be based on the real chromosome with the lowest cost in the real pool. At 140, a genetic algorithm is executed to generate at least one speculative chromosome. A speculative chromosome is an incremental modification of a value set associated with one or more parent chromosomes. The parent chromosomes can be real or speculative. At 150, the incremental cost function is executed to generate speculative costs associated with one or more speculative chromosomes. The speculative chromosomes and associated speculative costs can be stored in a speculative pool.

At 160, the methodology determines whether or not to perform a validation based on whether a predetermined validation criteria has been satisfied. The predetermined validation criteria can be based on a speculation count that corresponds speculative generations of one or more speculative chromosomes. Alternatively, the predetermined criteria can be based on a change in an incremental cost associated with a speculative chromosome generation, or a change in incremental cost associated with an incremental cost function exceeding a predetermined limit. The predetermined validation criteria can be based on speculative costs converging (e.g., not changing significantly). Furthermore, predetermined validation criteria can be based on a predetermined error level being exceeded, or an execution time limit associated with the genetic algorithm and the incremental cost function.

If the methodology determines a validation is desired (YES), the methodology returns to 100. At 100, a validation is initiated such that the real cost function is executed on one or more speculative chromosomes to generate real costs associated with the one or more speculative chromosomes, thus adding the one or more speculative chromosomes to the set of real chromosomes. If the methodology determines a validation is not desired (NO), the methodology returns to 140 to generate a new generation of speculative chromosomes and speculative costs at 150. The new generation of speculative chromosomes and associated speculative costs can be added to the speculative pool, such that new speculative chromosomes having lower speculative costs replace speculative chromosomes having higher speculative costs. The methodology then proceeds to 160 to determine if the validation criteria has been satisfied.

FIG. 8 illustrates an alternate methodology for selecting a value set associated with a set of parameters. At 200, real costs are generated for a plurality of first value sets represented as real chromosomes. The methodology then proceeds to 210. At 210, speculative costs are generated for a plurality of second value sets represented as speculative chromosomes. The speculative chromosomes represent value set variations of the first value set. At 220, it is determined if a predetermined validation has been satisfied. If a predetermined validation has not been satisfied (NO), the methodology proceeds to 240. At 240, at least one subsequent generation of speculative chromosomes are generated from parents selected from at least one of the plurality of speculative chromosomes and real chromosomes, and associated speculative costs are determined. The methodology then returns to 220. If a predetermined validation has been satisfied at 230 (YES), the methodology proceeds to 240. At 240, real costs are generated for at least one speculative chromosome of the plurality of speculative chromosomes.

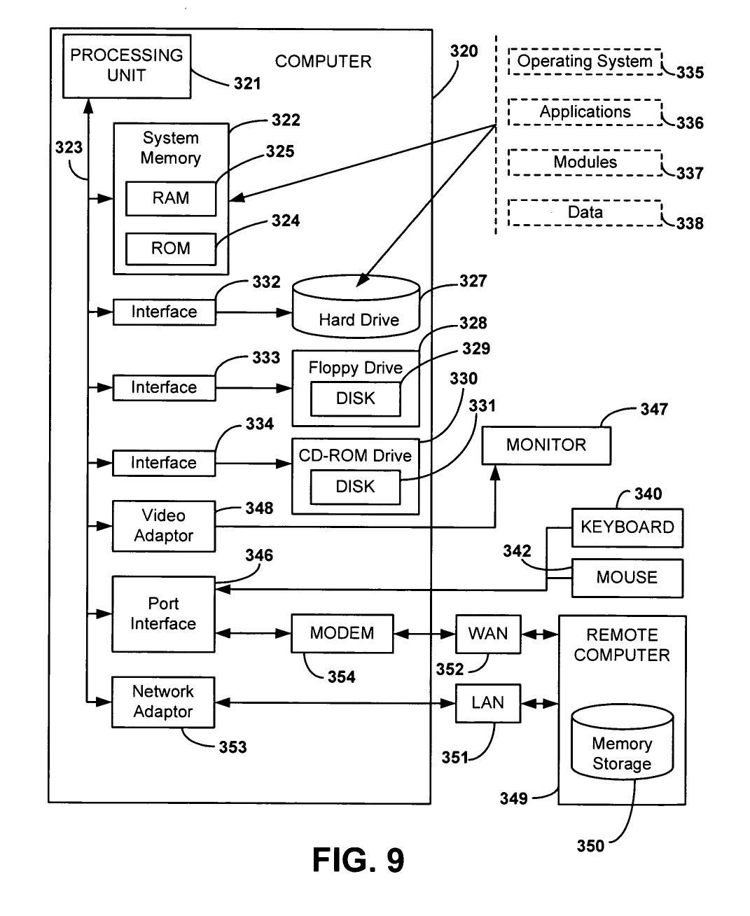

FIG. 9 illustrates a computer system 320 that can be employed to execute one or more embodiments employing computer executable instructions. The computer system 320 can be implemented on one or more general purpose networked computer systems, embedded computer systems, routers, switches, server devices, client devices, various intermediate devices/nodes and/or stand alone computer systems. Additionally, the computer system 320 can be implemented on various mobile clients such as, for example, a cell phone, personal digital assistant (PDA), laptop computer, pager, and the like.

The computer system 320 includes a processing unit 321, a system memory 322, and a system bus 323 that couples various system components including the system memory to the processing unit 321. Dual microprocessors and other multi-processor architectures also can be used as the processing unit 321. The system bus may be any of several types of bus structure including a memory bus or memory controller, a peripheral bus, and a local bus using any of a variety of bus architectures. The system memory includes read only memory (ROM) 324 and random access memory (RAM) 325. A basic input/output system (BIOS) can reside in memory containing the basic routines that help to transfer information between elements within the computer system 320.

The computer system 320 can includes a hard disk drive 327, a magnetic disk drive 328, e.g., to read from or write to a removable disk 329, and an optical disk drive 330, e.g., for reading a CD-ROM disk 331 or to read from or write to other optical media. The hard disk drive 327, magnetic disk drive 328, and optical disk drive 330 are connected to the system bus 323 by a hard disk drive interface 332, a magnetic disk drive interface 333, and an optical drive interface 334, respectively. The drives and their associated computer-readable media provide nonvolatile storage of data, data structures, and computer-executable instructions for the computer system 320. Although the description of computer-readable media above refers to a hard disk, a removable magnetic disk and a CD, other types of media which are readable by a computer, such as magnetic cassettes, flash memory cards, digital video disks and the like, may also be used in the operating environment, and further that any such media may contain computer-executable instructions.

A number of program modules may be stored in the drives and RAM 325, including an operating system 335, one or more application programs 336, other program modules 337, and program data 338. A user may enter commands and information into the computer system 320 through a keyboard 340 and a pointing device, such as a mouse 342. Other input devices (not shown) may include a microphone, a joystick, a game pad, a scanner, or the like. These and other input devices are often connected to the processing unit 321 through a corresponding port interface 346 that is coupled to the system bus, but may be connected by other interfaces, such as a parallel port, a serial port or a universal serial bus (USB). A monitor 347 or other type of display device is also connected to the system bus 323 via an interface, such as a video adapter 348.

The computer system 320 may operate in a networked environment using logical connections to one or more remote computers, such as a remote client computer 349. The remote computer 349 may be a workstation, a computer system, a router, a peer device or other common network node, and typically includes many or all of the elements described relative to the computer system 320. The logical connections can include a local area network (LAN) 351 and a wide area network (WAN) 352.

When used in a LAN networking environment, the computer system 320 can be connected to the local network 351 through a network interface or adapter 353. When used in a WAN networking environment, the computer system 320 can include a modem 354, or can be connected to a communications server on the LAN. The modem 354, which may be internal or external, is connected to the system bus 323 via the port interface 346. In a networked environment, program modules depicted relative to the computer system 320, or portions thereof, may be stored in the remote memory storage device 350.

What have been described above are examples of the present invention. It is, of course, not possible to describe every conceivable combination of components or methodologies for purposes of describing the present invention, but one of ordinary skill in the art will recognize that many further combinations and permutations of the present invention are possible. Accordingly, the present invention is intended to embrace all such alterations, modifications and variations that fall within the spirit and scope of the appended claims.

1. A method comprising:
determining real costs for a plurality of first value sets represented as a plurality of real chromosomes;
determining speculative costs for a plurality of second value sets represented as a plurality of speculative chromosomes, the speculative chromosomes representing value set variations of the first value sets; and
postponing validation of speculative chromosomes by generating subsequent generations of speculative chromosomes and associated speculative costs from parents selected from at least one of the plurality of real chromosomes and the plurality of speculative chromosomes, until a predetermined validation criteria has been satisfied.

2. The method of claim 1, determining real costs for at least one speculative chromosome of the plurality of speculative chromosomes when the predetermined validation criteria has been satisfied.

3. The method of claim 1, further comprising assigning a speculation count to each generation of speculative chromosomes, the predetermined validation criteria being a specific speculation count.

4. The method of claim 1, the determining real costs comprising executing a real cost function on the plurality of real chromosomes and the determining speculative costs comprising executing an incremental cost function on the plurality of speculative chromosomes.

5. The method of claim 1, further comprising assigning a real cost to the plurality of real chromosomes based on the minimum cost real chromosome in the plurality of real chromosomes and assigning a speculation cost to each generation of speculative chromosomes based on the minimum cost speculative chromosome in a respective generation.

6. The method of claim 5, the predetermined validation criteria comprises a speculative cost difference between a generation of speculative chromosomes and a subsequent generation of speculative chromosomes exceeding a predetermined cost change limit.

7. The method of claim 5, the predetermined validation criteria comprises a cost difference between a generation of speculative chromosomes and the plurality of real chromosomes exceeding a predetermined cost change limit.

8. The method of claim 1, the predetermined validation criteria comprises speculative costs converging for subsequent generations of speculative chromosomes.

9. The method of claim 1, the predetermined validation criteria comprises speculation errors associated with each generation of speculation exceeding a predetermined limit.

10. The method of claim 1, the predetermined validation criteria comprises exceeding an execution time limit for generating subsequent generations of speculative chromosomes and generating speculative costs associated with the subsequent generations.

11. A computer-readable medium having computer executable instructions for performing a method comprising:
executing a real cost function on a plurality of first value sets represented as a plurality of real chromosomes to generate a plurality of real costs for each of the plurality of real chromosomes;
executing a genetic algorithm to generate a plurality of speculative chromosomes, the speculative chromosomes representing value set variations of the first value sets;
executing an incremental cost function on the plurality of speculative chromosomes to generate a plurality of speculative costs for each of the plurality of speculative chromosomes; and
repeating execution of the genetic algorithm to produce subsequent generations of speculative chromosomes and repeating execution of the incremental cost function on subsequent generations to provide speculative costs for the subsequent generations of speculative chromosomes, until a predetermined validation criteria has been satisfied.

12. The method of claim 11, further comprising generating the incremental cost function based on at least one real chromosome and associated real cost.

13. The method of claim 11, further comprising validating at least one speculative chromosome of the plurality of speculative chromosomes when the predetermined validation criteria has been satisfied, the validating at least one speculative chromosome comprising executing the real cost function on the at least one speculative chromosome to generate a real cost associated with at least one speculative chromosome.

14. The method of claim 13, further comprising repeating the execution of the genetic algorithm to generate a plurality of new speculative chromosomes from the at least one validated speculative chromosome and executing a new incremental cost function on the plurality of new speculative chromosomes to generate a plurality of speculative costs for each of the plurality of new speculative chromosomes.

15. The method of claim 11, the predetermined validation criteria is based on at least one of satisfying a speculative chromosome generation count, exceeding a predetermined cost change limit between speculative generations and exceeding a predetermined cost change limit between the plurality of real chromosomes and a speculative generation.

16. A system comprising:
a real cost function that generates real costs for each of a plurality of value sets represented as a plurality of real chromosomes;
an incremental cost function that generates a plurality of speculative costs corresponding to a plurality of value set variations of at least one of the plurality of real chromosomes, the plurality of value set variations represented as a plurality of speculative chromosomes; and
a validator that initiates a validation on at least one speculative chromosome upon satisfaction of a predetermined validation criteria, a validation comprising executing the real cost function on the at least one speculative chromosome to generate a real cost associated with at least one speculative chromosome.

17. The system of claim 16, further comprising a genetic algorithm that generates the plurality of speculative chromosomes from parent chromosomes selected from the real chromosomes.

18. The system of claim 17, the genetic algorithm generates at least one subsequent generation of speculative chromosomes employing parents chromosomes selected from at least one of real chromosomes and speculative chromosomes.

19. The system of claim 16, the real cost function comprising an optimization tool for optimizing a circuit design, and the plurality of value sets being a plurality of circuit configurations generated by the optimization tool.

20. The system of claim 16, the predetermined validation criteria is based on at least one of satisfying a speculative chromosome generation count, exceeding a predetermined cost change limit between speculative generations and exceeding a predetermined cost change limit between the plurality of real chromosomes and a speculative generation.

21. A system for minimizing a cost associated with a set of parameters representing a solution, the system comprising:
means for determining real costs associated with a plurality of real chromosomes representing different value sets associated with a set of parameters;
means for generating a plurality of speculative chromosomes from parent chromosomes selected from at least one of the plurality of speculative chromosomes and the plurality of real chromosomes;
means for determining a speculative cost for a respective speculative chromosome based on a cost of at least one parent chromosome and a difference in value sets of the at least one parent chromosome and the respective speculative chromosome; and
means for postponing validation of the plurality of speculative chromosomes until a predetermined validation criteria has been satisfied.

22. The system of claim 21, the means for generating speculative chromosomes being operative to generate additional generations of speculative chromosomes from parents selected from the at least one of the plurality of speculative chromosomes and the plurality of real chromosomes.

23. The system of claim 21, the predetermined validation criteria is based on at least one of satisfying a speculative chromosome generation count, exceeding a predetermined cost change limit between speculative generations and exceeding a predetermined cost change limit between the plurality of real chromosomes and a speculative generation.

24. The system of claim 21, validation of the plurality of speculative chromosomes comprising executing the means for determining a real cost on at least one speculative chromosome.
